(19) United States
(12) Patent Application Publication
(10) Pub. No.: US 2006/0178635 A1
(43) Pub. Date: Aug. 10, 2006

(54) Easy entry catheters

(76) Inventor: George Hadley Callaway, Raleigh, NC (US)

Correspondence Address: HUTCHISON LAW GROUP PLLC, PO BOX 31686, RALEIGH, NC, 27612, US

(21) Appl. No.: 11/053,058

(22) Filed: Feb. 8, 2005

(51) Int. Cl. A61M 5/178

(52) U.S. Cl. 604/164.09; 604/506

(57) ABSTRACT

Catheter devices and methods for easy insertion of same. The assembly may include a needle having a patient insertion end and a second end, a length and a diameter, a first catheter, the first catheter having a patient insertion end and a second end, a length, and an inner diameter sufficient to slidably receive at least a portion of the length of the needle within the first catheter, a second catheter, the second catheter having a patient insertion end and a second end, a length, and an inner diameter sufficient to slidably receive at least a portion of the length of the first catheter within the second catheter, and wherein the first catheter is circumferentially disposed about at least a portion of the needle, the second catheter is circumferentially disposed about at least a portion of the first catheter, and the first and second catheters are independently slidable from one another and the needle

### FIELD

The invention generally relates to catheters and methods of using the same.

### BACKGROUND

Various catheter insertion assemblies are known. For example, methods of insertion of catheters include within-the-needle, over-the-needle, and over-a-guide-wire methods. Some techniques involve a combination of methods.

Within-the-needle methods limit the diameter of the catheter and usually retain the needle over the catheter after insertion. The needle tip is inserted into the vessel and the catheter is passed through the needle into the vessel. The needle is withdrawn and stored on the catheter outside the patient. Within-the-needle methods have been largely abandoned in practice.

Over-the-needle methods allow larger catheter diameters but are difficult in small diameter blood vessels. The needle tip is inserted into the vessel and the catheter is advanced off the needle into the blood vessel. The needle is withdrawn and discarded appropriately. Over-the-needle methods are widely used, especially for peripheral veins. Most recent innovation in over-the-needle methods is in the area of safe handling of the needle.

Over-a-guide-wire techniques involve placement of the guide wire through the needle and subsequent placement of the catheter over the guide wire. This combines the advantages of the within- and over-the-needle methods but requires a cumbersome extra step of placement of the guide wire. Over-a-guide-wire methods are generally used for arterial and central venous access, not peripheral veins.

An example of a within-the-needle device is found in U.S. Pat. No. 4,068,659, the subject matter of which is herein incorporated by reference, which describes a catheter containing a stiffening wire, which is inserted within-the-needle.

Various examples of over-the-needle designs exist including U.S. Pat. No. 4,411,654, subject matter of which is herein incorporated by reference, which describes a peel-away-type introducer, which is inserted over the needle. The catheter is then passed through the introducer into the vein, and the introducer is peeled off the catheter into two pieces. U.S. Pat. No. 5,007,901, the subject matter of which is herein incorporated by reference, describes a catheter inserted over the needle, with a mechanism for protection of the withdrawn needle.

An example of an over-a-guidewire device is included in U.S. Pat. No. 4,417,886, the subject matter of which is herein incorporated by reference. U.S. Pat. No. 4,772,264, the subject matter of which is herein incorporated by reference, describes a guide tube which functions as a guide wire. The guide tube is inserted within-the-needle, and the catheter is advanced over the guide tube.

U.S. Pat. No. 5,391,152, the subject matter of which is herein incorporated by reference, describes an introducer assembly which is used to establish access to a central vein. In this method, a needle is used to enter the vein and pass a guidewire, over which the introducer assembly is passed. The introducer assembly is really two catheters, one inside the other. The inner catheter is relatively rigid and is called a dilator. The outer catheter is a large-diameter tube which remains in the vein after the dilator and guide wire are removed. This device is problematic in that it still requires the extra cumbersome step of using a guidewire.

### SUMMARY

A catheter insertion assembly is disclosed including a needle having a patient insertion end and a second end, a length and a diameter, a first catheter, the first catheter having a patient insertion end and a second end, a length, and an inner diameter sufficient to slidably receive at least a portion of the length of the needle within the first catheter, a second catheter, the second catheter having a patient insertion end and a second end, a length, and an inner diameter sufficient to slidably receive at least a portion of the length of the first catheter within the second catheter, and wherein the first catheter is circumferentially disposed about at least a portion of the needle, the second catheter is circumferentially disposed about at least a portion of the first catheter, and the first and second catheters are independently slidable from one another and the needle.

Also disclosed is a catheter insertion assembly including a needle having a distal end and a proximal end, a first catheter partially enclosing the needle and having a distal end, a proximal end, and an outer diameter greater than the outer diameter of the needle, a second catheter partially enclosing the needle and the first catheter and having a distal end, a proximal end, and an outer diameter greater than the outer diameter of the first catheter and wherein the distal end of the needle, and the distal ends of the first and second catheters are configured that upon insertion into a patient, the needle punctures the patient first, followed by the first catheter and then the second catheter, thereby sequentially increasing the diameter of the puncture in the patient.

Methods of inserting catheters into a patient are also disclosed including:
a. providing a needle having a patient insertion end and a second end, a length and a diameter,
b. providing a first catheter, the first catheter having a patient insertion end and a second end, a length, and an inner diameter sufficient to slidably receive at least a portion of the length of the needle within the first catheter wherein the patient insertion of the needle is distal to the patient insertion end of the first catheter,
c. providing a second catheter, the second catheter having a patient insertion end and a second end, a length, and an inner diameter sufficient to slidably receive at least a portion of the first catheter wherein the patient insertion ends of the needle and the first catheter are distal to the patient insertion end of the second catheter and the first and second catheters are independently slidable from one another and the needle,
d. inserting the patient insertion ends of the needle, first catheter and second catheter into a patient, and;
e. sliding the needle out of the second end of the first catheter.

Another method disclosed includes:
a. providing a needle having a distal end and a proximal end,
b. providing a first catheter partially enclosing the needle and having a distal end, a proximal end, and an outer diameter greater than the outer diameter of the needle,
c. providing a second catheter partially enclosing the needle and the first catheter and having a distal end, a proximal end, and an outer diameter greater than the outer diameter of the first catheter, wherein the distal end of the needle, and the distal ends of the first and second catheters are configured that upon insertion into a patient, the needle punctures the patient first, followed by the first catheter and then the second catheter, thereby sequentially increasing the diameter of the puncture in the patient
d. inserting the distal ends of the needle, first and second catheters into a patient,
e. removing the needle from the first catheter, and;
f. removing the first catheter from the second catheter.

Another method disclosed includes:
a. providing a needle having a distal end and a proximal end,
b. providing a first catheter partially enclosing the needle and having a distal end, a proximal end, and an outer diameter greater than the outer diameter of the needle,
c. providing a second catheter partially enclosing the needle and the first catheter and having a distal end, a proximal end, and an outer diameter greater than the outer diameter of the first catheter, wherein the distal end of the needle, and the distal ends of the first and second catheters are configured that upon insertion into a patient, the needle punctures the patient first, followed by the first catheter and then the second catheter, thereby sequentially increasing the diameter of the puncture in the patient
d. inserting the distal ends of the needle, first and second catheters into a patient,
e. advancing the catheters farther into the patient and off of the needle, and;
f. advancing the second catheter farther into the patient and off of the first catheter, and;
g. removing both the needle and the first catheter.

### BRIEF DESCRIPTION OF THE FIGURES

FIG. 1 depicts a catheter insertion assembly.

FIG. 2 is a magnified view of the distal end of the catheter insertion assembly.

FIG. 3 is a cross section view of a catheter insertion assembly.

FIG. 4 is a magnified cross section view of the distal end of a catheter insertion assembly.

FIG. 5 is a catheter insertion assembly with the three major parts disassembled from each other.

FIG. 6 is a catheter insertion assembly demonstrating the catheters being advanced off the needle.

FIG. 7 is a catheter insertion assembly demonstrating the outer catheter being advanced off the inner catheter.

FIG. 8 depicts the outer catheter alone, which would be left indwelling in the patient after the inner catheter and needle have been removed.

FIG. 9 depicts a catheter assembly with one type of needle safety device comprised of a clip within the hub of the inner catheter which captures the end of the needle as it is withdrawn from the inner catheter.

FIG. 10 depicts a catheter insertion assembly with a second type of needle safety device in the before-use configuration.

FIG. 11 depicts a catheter insertion assembly with the second type of needle safety device after use, with the needle contained within a cylindrical needle-guard.

FIG. 12 depicts a catheter insertion assembly with a needle safety device after use, with the outer catheter separated from the combined inner catheter and safety device.

FIG. 13 is a cross section view of a catheter insertion assembly with a needle safety device, prior to use.

FIG. 14 is a sectioned detail view of the middle hub of a catheter assembly with a safety device demonstrating that the inner catheter hub has been cemented or joined with the cylindrical needle guard.

FIG. 15 is a sectioned view of a catheter assembly with a safety device with the needle in a protected position within the cylindrical needle guard.

FIG. 16 is a sectioned detail view of the middle hub of a catheter assembly with a safety device demonstrating how the needle tip is contained within the hub of the inner catheter when the assembly is in the protected position.

### DETAILED DESCRIPTION

The devices and methods disclosed herein relate to easy entry catheters and methods of using the same. The devices and methods may also be used to provide access to other anatomic structures as described herein. The devices and methods have numerous advantages including allowing for large diameter catheters to be inserted less traumatically into smaller vessels while avoiding the need for a cumbersome guide wire. Large bore catheters may be inserted into the lumen of small vessels ducts or tubes.

Devices and methods disclosed herein may be used, for example, for all intravascular applications including central veins such as the antecubital, internal jugular and subclavian veins. The devices and methods may also be used for introduction of percutaneous billiary duct cannulation and percutaneous nephrostomy. The device and methods may also be useful for cannulation of bile ducts, pancreatic ducts, urinary tract, lymphatic vessels, epidural space, and fluid channels of the central nervous assembly. The devices and methods disclosed may also be used to enter other spaces in the patient such as fluid filled cavities in the brain and the epidural space of the spine. The devices and methods may also be used for drainage, insertion of dyes for imaging (such as cardiac imaging), introducing fluids, administering anesthetics, insertion of optical devices or scopes, chest tube drains, and pericardial taps.

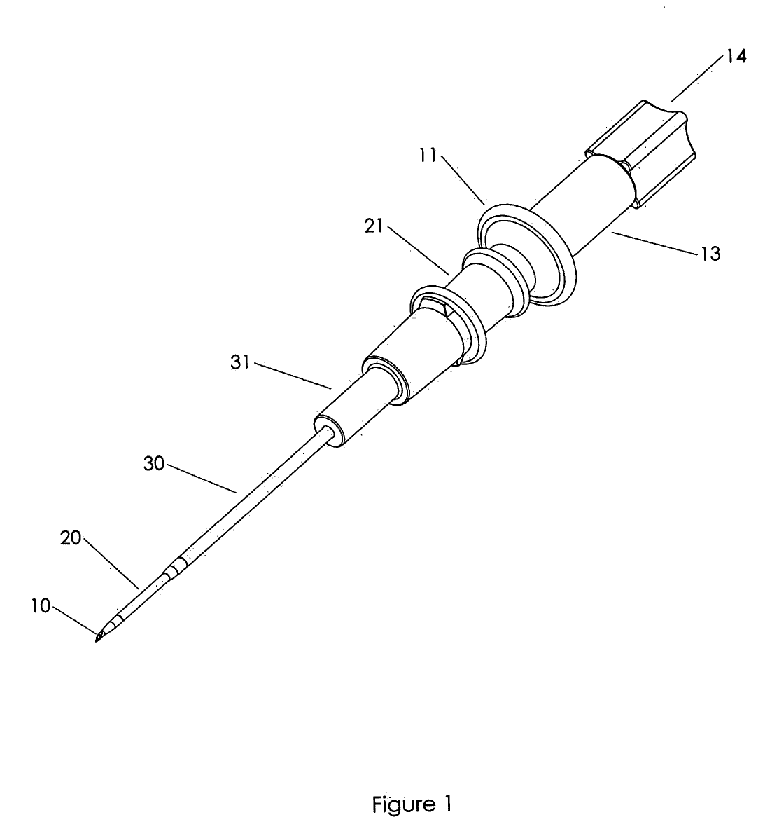

The catheters disclosed herein involve the use of a small diameter catheter to act as a guide and dilator for a catheter of larger diameter. The devices include, for example as depicted in FIGS. 1, 2, 3 and 4, a hollow needle (10) with a sharp distal end (12) and a proximal end connected to a hub (11) and flash-back chamber (13 and 14). The devices also include a small-diameter catheter (20) connected to a hub (21), and a large-diameter catheter (30) connected to a hub (31). The patient insertion end or distal end (22) of the small catheter and the patient insertion end or distal end (32) of the large catheter may be beveled or tapered which is depicted in the drawings as parallel arcs in drawings 1 and 5-12. The distal end (12) of the needle (10) protrudes from the end of the small-diameter catheter (20), and the small-diameter catheter (20) protrudes from the large-diameter catheter (30). The small-diameter catheter (20) has inner diameter sufficient to accommodate insertion of the needle (10) through the small-diameter catheter (20) and outer diameter sufficient to permit insertion of the small-diameter catheter (20) into the large-diameter catheter (30). Both catheters may be flexible but not necessarily to the same degree.

FIG. 1 is shows a catheter insertion assembly as described above.

FIGS. 1, 2, 3 and 4 show a catheter assembly without a needle safety device. A proximal hub (11) is attached to a hollow needle (10) which extends beyond the ends of the catheters (20 and 30). The proximal hub (11) contains a clear flash-chamber (13 and 14) which allows visualization of blood when the tip of the needle enters a blood vessel. The flash chamber (13) has a porous plug (14) containing a material such as cotton or cellulose which allows air to exit the flash chamber (13) when the blood enters, but which retains the blood within the chamber. The middle hub (21) is attached to an inner catheter (20) which is slightly shorter than the needle (10) and longer than the outer catheter (30). The inner catheter (20) has inner diameter which is slightly larger than the outer of the needle (10). The inner catheter (20) has outer diameter which is slightly smaller than the inner diameter of the outer catheter (30). The distal hub (31) is attached to an outer catheter (30) which is slightly shorter than the inner catheter (20). The outer catheter (30) has inner diameter slightly larger than the outer diameter of the inner catheter (20). The needle (10) has a sharp beveled insertion end (12). The catheters have tapered ends (22 and 32) to enlarge the hole in a blood vessel as the assembly is inserted. The distal hub (31) and middle hub (21) have fittings which engage with and attach to standard intravenous tubing.

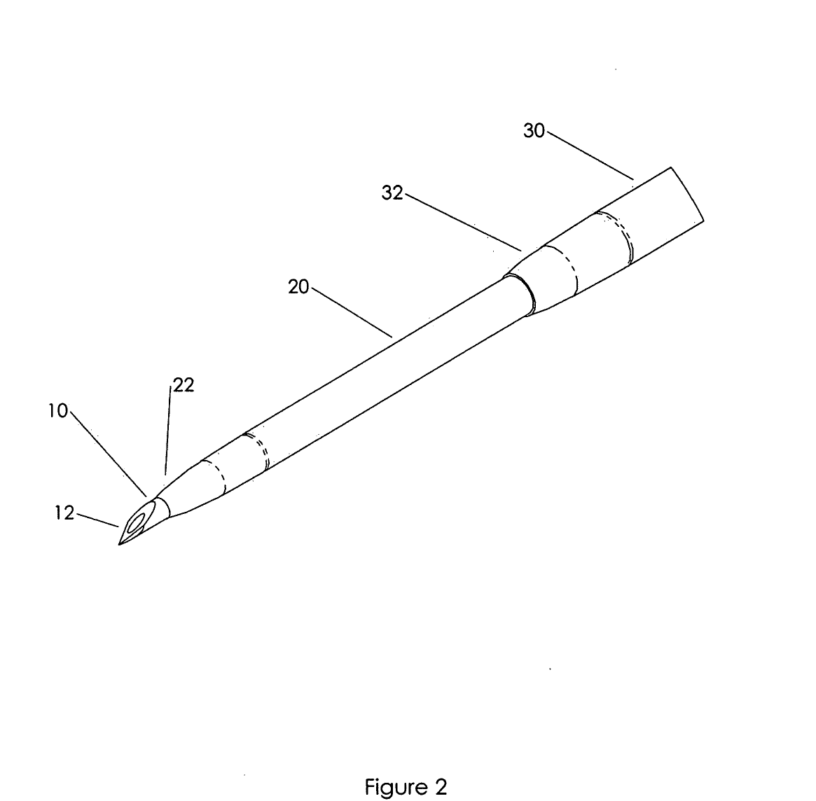

FIG. 2 shows a detail of the insertion end or distal end of the catheter assembly as described above.

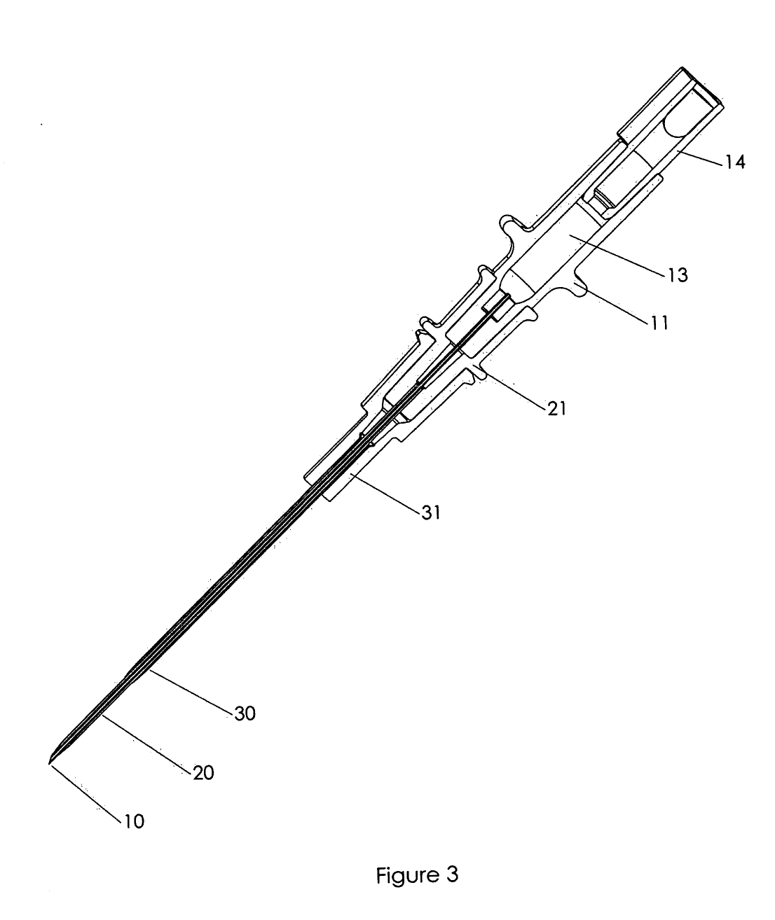

FIG. 3 shows a cut-away section of a catheter insertion assembly. Three hubs are shown, one inside the other. The proximal hub (11) is connected to a hollow needle (10) on one end and to a clear flash-chamber (13) on the other end. The middle hub (21) is connected to the inner catheter (20) on one end and is open on the other end. The distal hub (31) is connected to the outer catheter (30) on one end and is open on the other end. After assembly, the hollow needle (10) is inside the inner catheter (20) which is inside the outer catheter (30). The hubs fit together with slight friction which prevents unintentional separation. The hollow needle (10) has a sharp, beveled end (12) which protrudes beyond the catheters. The inner catheter (20) has a tapered tip (22) which extends beyond the outer catheter. The outer catheter (30) has a tapered tip (32). The inner catheter (20) has diameter and wall thickness to “make up” the difference between the outer diameter of the needle (10) and the inner diameter of the outer catheter (30).

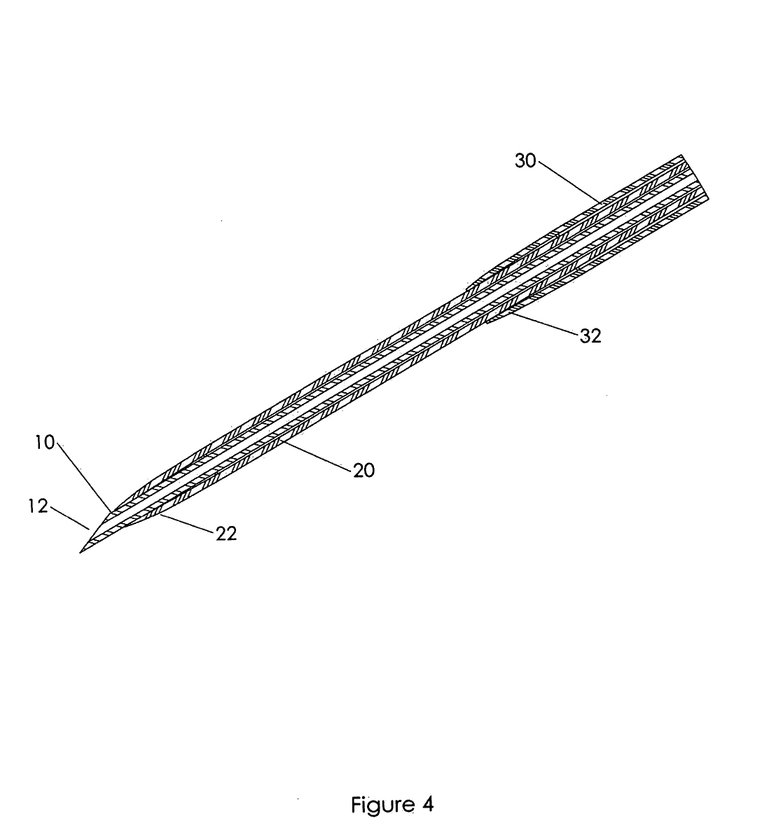

FIG. 4 is a cut away section of the distal or insertion end of the catheter assembly. The hollow needle (10) and outer catheter (30) are separated by the inner catheter (20), which has diameter and wall thickness to “make up” the difference between the outer diameter of the needle and the inner diameter of the outer catheter. The interfaces between the three parts allow easy sliding. The hollow needle (10) has a sharp, beveled end (12) which protrudes beyond the catheters. The inner catheter (20) has a tapered tip (22) which extends beyond the outer catheter (30). The outer catheter (30) has a tapered tip (32).

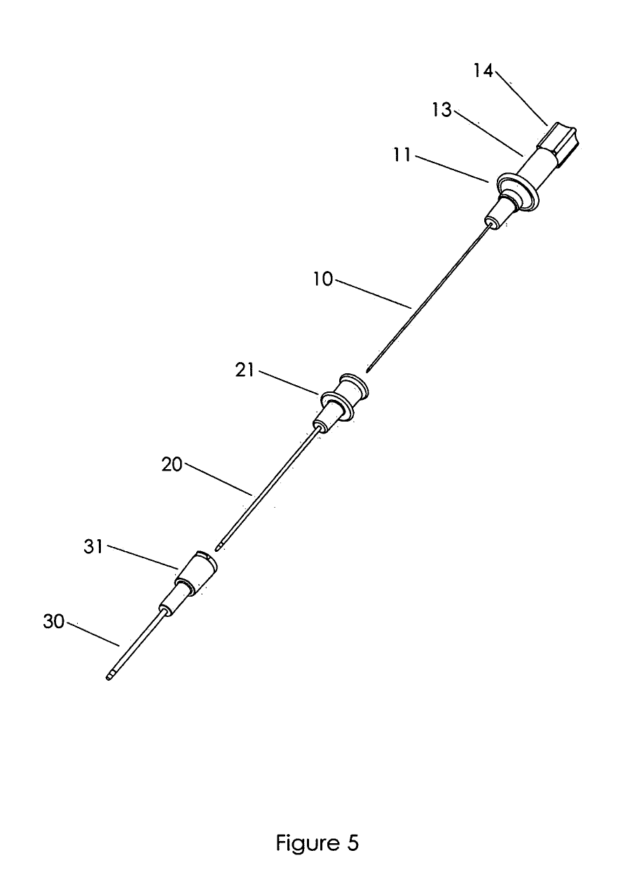

FIG. 5 shows the three major parts of the assembly separated along their common axis. The needle (10), proximal hub (11) and flash chamber (13) are on the right. The needle is used in the insertion process to penetrate the skin and soft tissues and to locate the fluid collection or blood vessel. The small catheter (20) and its hub (21) in the center are used in the insertion process to dilate the puncture wound. The outer catheter (30) and its hub (31) on the left are secured to and left indwelling in the patient and used to administer or drain fluids.

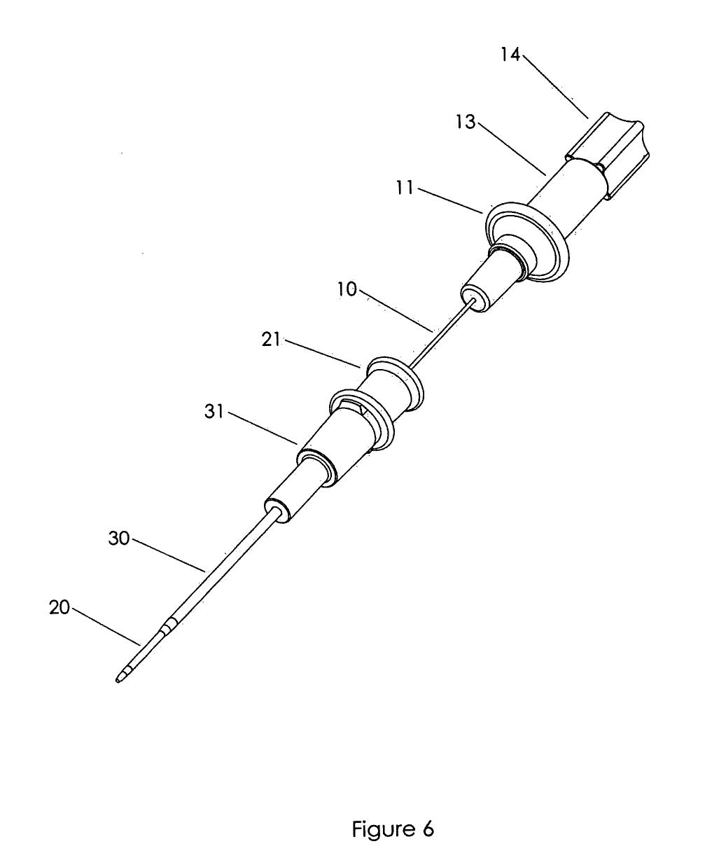

FIG. 6 shows the catheter assemblies after the catheters (20 and 30) and their hubs (21 and 31) have been advanced partially off the needle (10).

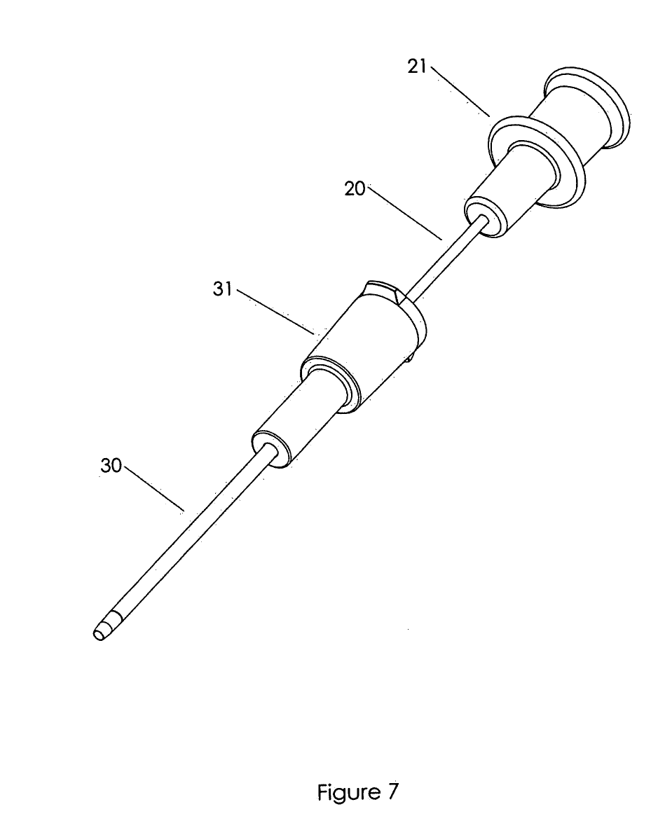

FIG. 7 shows the outer catheter (30) and its hub (31) after it has been partially been advanced off the inner catheter (20).

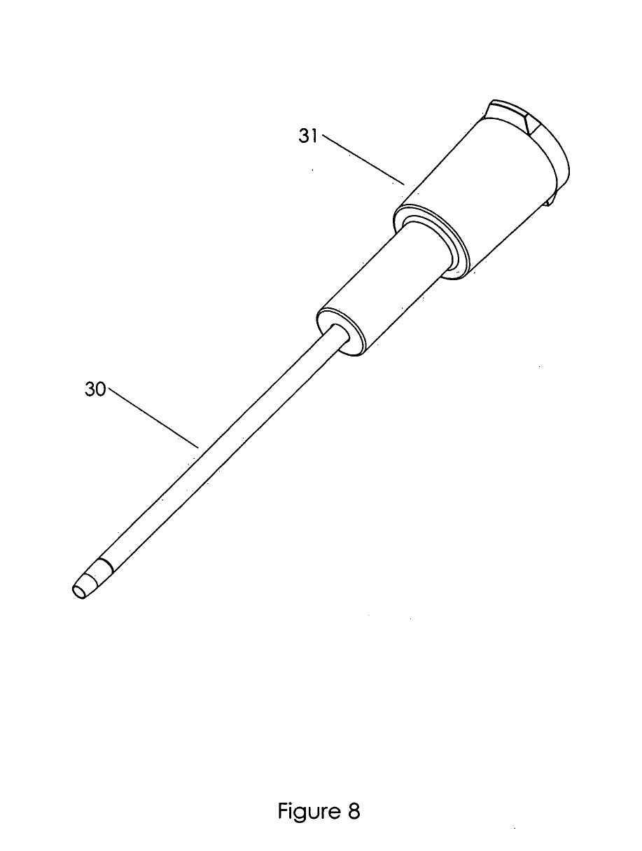

FIG. 8 depicts the outer catheter (30) and its hub (31) alone. It shows the catheter as it is ready for use after completion of the insertion process.

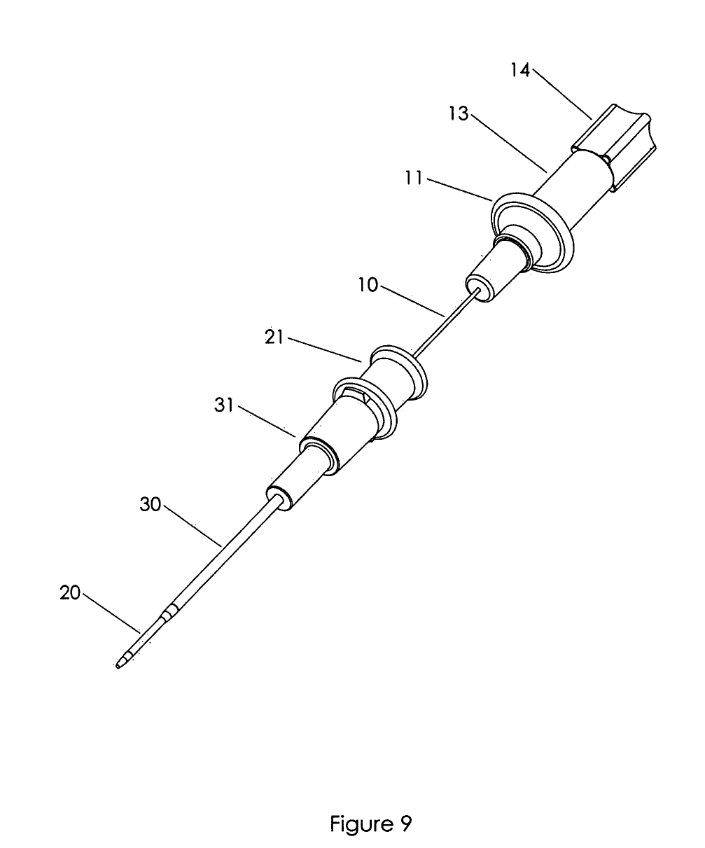

FIG. 9 shows the catheter assembly with a needle safety device disclosed in U.S. Pat. No. 6,616,630, hereby incorporated by reference in the entirety. FIG. 9 illustrates how the catheter assembly could be integrated with an existing needle protection device. In FIG. 9 the catheters (20 and 30) and their hubs (21 and 31) have been partially advanced off the needle (10). This needle safety device includes a metal clip in the hub (21) of the inner catheter which, upon withdrawal of the needle (10), captures and contains the needle tip within the hub (21) of the inner catheter. The clip and hub (21) protect users from the sharp tip of the needle (10). In one version, the needle (10) and attached clip could be withdrawn from the hub (21). In the preferred version, the inner catheter (20), its hub (21) and the needle (10) remain attached together and are discarded together in a safe manner.

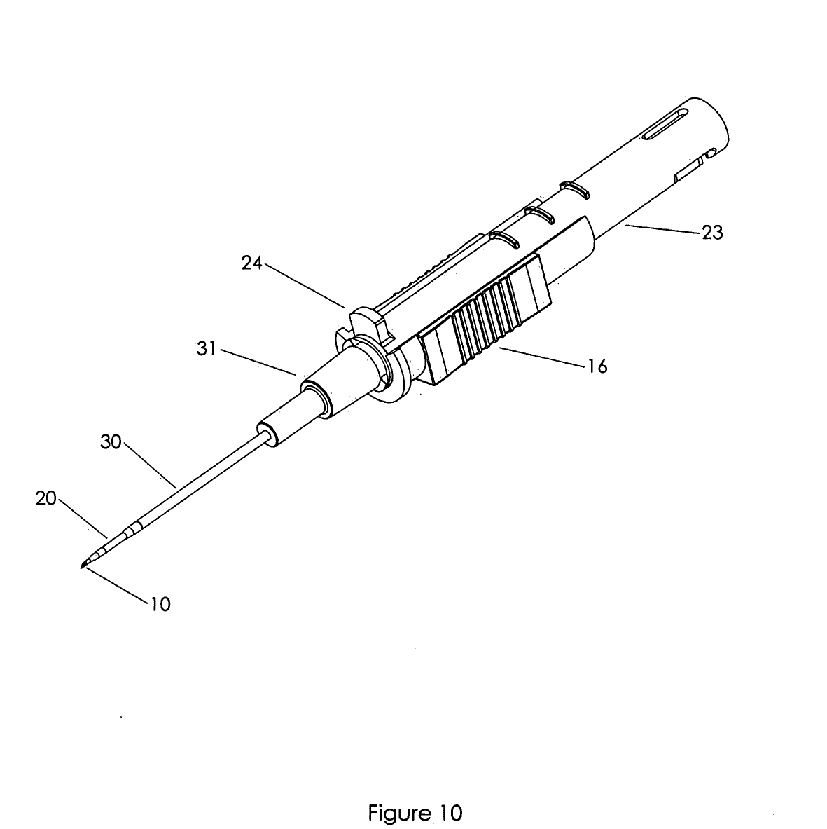

FIGS. 10 through 16 show the catheter assembly with a needle safety system disclosed in U.S. Pat. Nos. 5,000,740 and 5,092,845, hereby incorporated by reference in their entirety. FIGS. 10 through 16 illustrate how the catheter assembly could be integrated with an existing needle protection device. The needle (10) is attached to a modified flash chamber (15), which has finger-grip extensions (16) with texturing for handling. The inner catheter (20) is attached by a tapered hub (21) to an elongated cylindrical needle-guard (23) which is clear to allow visualization of the flash chamber (15) inside it. The outer catheter (30) and its hub (31) are unchanged from the prior version. In use, the needle (10) is advanced until it enters a blood vessel and blood is seen in the flash-chamber (15). The two catheters (20 and 30) are then advanced together off the needle (10) into the lumen of the blood vessel. To do this, the user holds the flash chamber extensions (16) in a fixed position, while pushing on flange (24) of the cylinder (23) to advance the inner (20) and outer (30) catheters together. As the cylinder (23) and catheters (20 and 30) are advanced, the cylinder (23) contains more and more of the needle (10). When the catheters (20 and 30) are fully advanced off the needle (10), the flash chamber (15) and cylinder (23) snap into a locked configuration that contains the needle tip (12) within the hub (21) of the inner catheter and the needle shaft (10) and a portion of the flash chamber (15) within the cylindrical needle guard (23). In the final insertion step, the outer catheter (30) is advanced off the inner catheter (20), as in all other versions of the technique. The inner catheter (20), hub (21), cylinder (23), needle (10) and flash chamber (15), which have been irreversibly snapped together, may be safely discarded as a single unit.

FIG. 10 shows a catheter assembly with a needle safety device, ready for use.

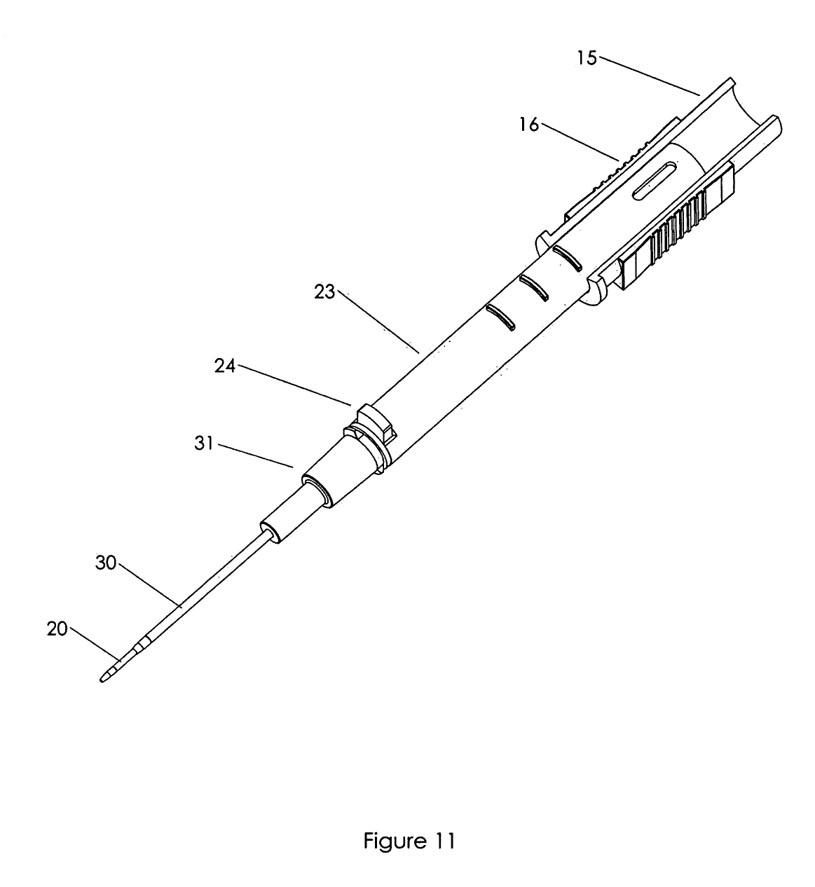

FIG. 11 shows a catheter assembly with a needle safety device, after the catheters (20 and 30) have been advanced off the needle (10) and with the needle captured within the cylindrical needle guard (23).

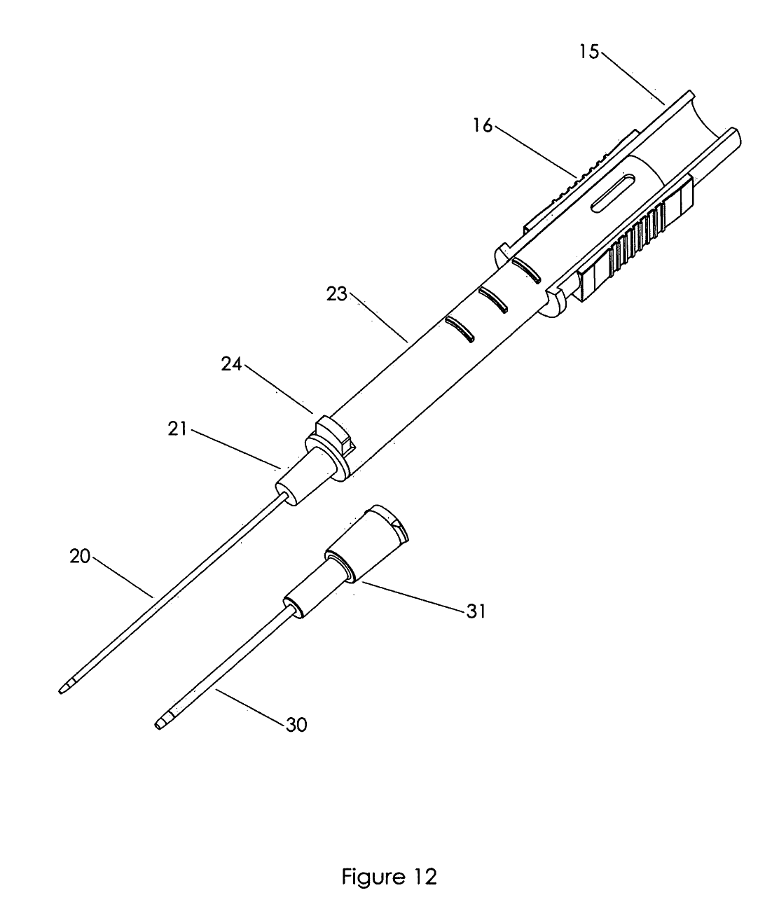

FIG. 12 show a catheter assembly with a needle safety device as in FIG. 11, except the outer catheter (30) has been advanced off the inner catheter (20). The inner catheter and safety device, shown at top, form a unit which cannot be disassembled and should be discarded after use. The outer catheter (30) and its hub (31), shown below, remains attached to the patient and is used to administer or withdraw fluids.

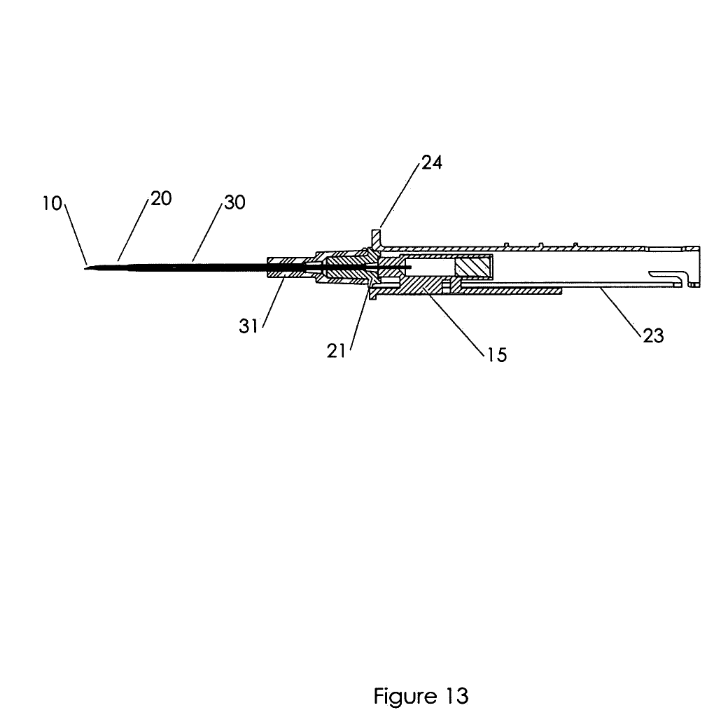

FIG. 13 is a cut-away section of a catheter assembly with a needle safety device. The needle (10) is attached to a flash chamber (15). The inner catheter (20) is attached by a tapered hub (21) to a cylindrical needle-guard (23) which is clear to allow visualization of the flash chamber (15) inside it. The outer catheter (30) and its hub (31) are unchanged from the prior version.

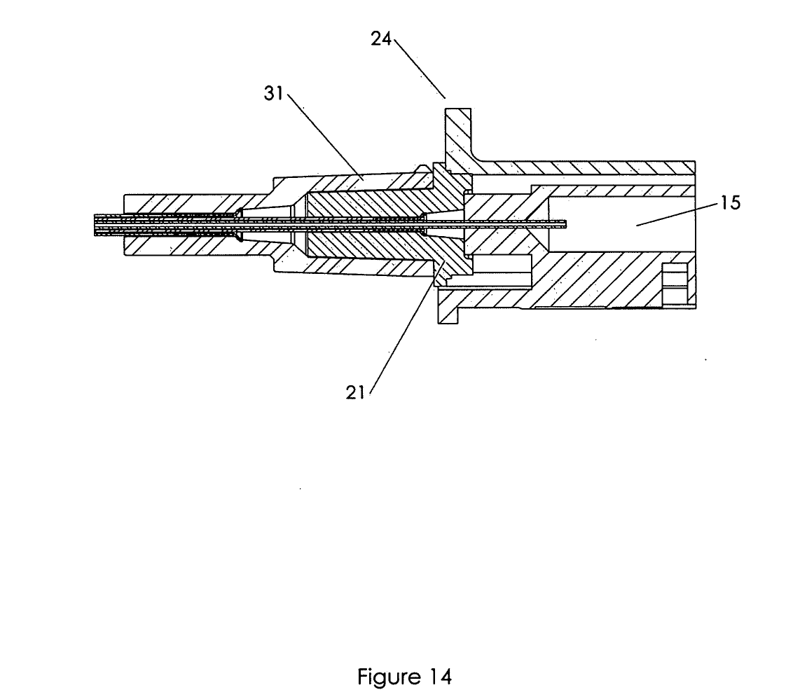

FIG. 14 is a detail of a cut-away section of a catheter assembly with a needle safety device. This figure illustrates how the middle hub (21) is joined by cement or other attachment means to the cylindrical needle-guard (23). The flash chamber (15) and its finger-grip extensions (16) are connected to the needle. The distal hub (31) fits over the tapered middle hub (21).

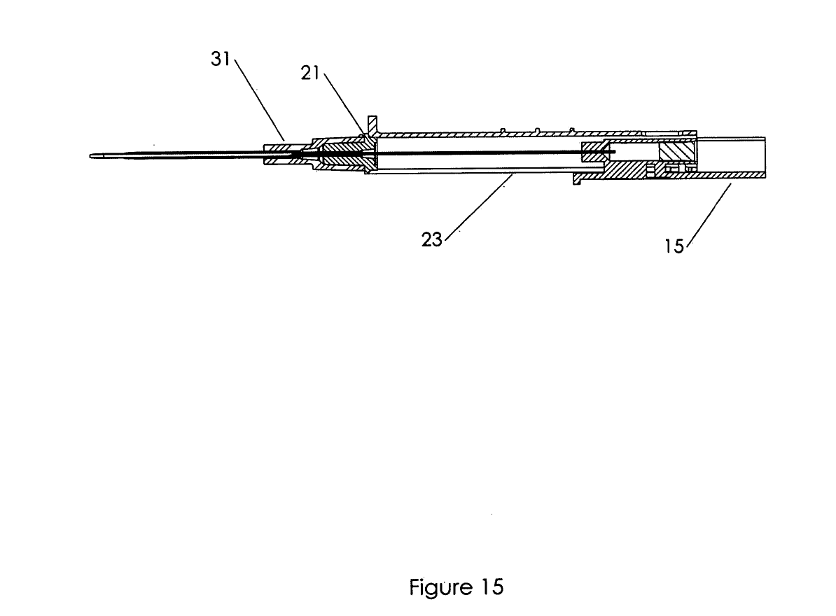

FIG. 15 is a cut-away section of a catheter assembly with a needle safety device in the “safe” position. This view demonstrates how the middle hub (21) and clear cylindrical needle-guard (23) contain the needle and some of the flash chamber (15) after both catheters (20 and 30) have been deployed off the needle. An extension of the flash chamber (15) snaps into an opening in the cylinder (23) to hold the needle tip inside the tapered hub (21) which joins the inner catheter and cylinder (23). At this stage in the insertion process, both catheters would be inside the blood vessel. The next step would be to advance the outer catheter (30) and its hub (31) off the inner catheter (20).

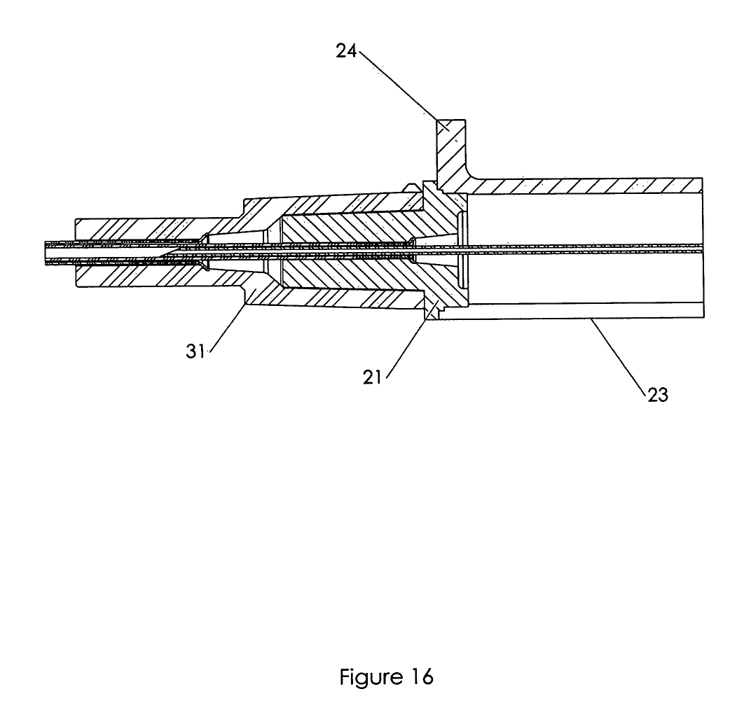

FIG. 16 is a detail of a cut-away section of a catheter assembly with a needle safety device in the “safe” position. This figure shows how the tip of the needle is contained within the middle hub (21) after the flash chamber (15) has been snapped into the “safe” position relative to the clear cylindrical needle-guard (23). The outer catheter (30) and distal hub (31) are shown in their initial position, before they are advanced off the inner catheter (20).

In use, the assembly is advanced until the needle enters, for example, a blood vessel. The small- and large-diameter catheters are advanced off the needle into the vessel together. The soft tip of the small-diameter catheter can pass within the vessel more easily and less traumatically than either the needle or large-diameter catheter could pass. It guides the larger catheter into the vessel. When a sufficient length of the catheters has been sufficiently inserted into the patient and a length of the catheters remain outside the patient, the large-diameter catheter is advanced off the small diameter catheter. The needle and small diameter catheter are withdrawn and discarded. In one embodiment, the hub of the small-diameter catheter may shield the sharp end of the needle.

The outer catheter may be used to hold open access to the blood vessel or lumen, so that the inner catheter and needle can be removed and replaced by a different catheter (i.e. a much longer one). The outer catheter may incorporate an end-diaphragm, side port, or other means to control access to the vessel. The outer catheter may be scored down its sides, to make possible peeling apart the outer catheter for removal in two parts while leaving the inner catheter in the vessel.

The needle may protrude beyond the end of the small diameter catheter and the small diameter catheter may protrude beyond end of the large diameter catheter. A distance of 2-4 mm and 3-6 mm is preferred respectively.

The devices may be constructed of biocompatible materials typically used in the formation of catheters and syringes such as various polyurethanes and other polymers capable of being processed into shaped articles. Examples include silicone, polyurethane, and fluoropolymers such as Teflon—FEP, PFA, or PTFE. Polyurethane, such as the Becton-Dickenson material Vialon, may be preferred over Teflon for peripheral IVs because it is stiff and slick for easy insertion, and softens once in the vein for a period of time, which leads to fewer complications like phlebitis. The materials may be optically transparent. For percutaneously inserted central catheters (PICC lines) and other longer-term and long-length catheters, the preferred materials are silicone and polyurethane. One or more of the catheters may be rigid or made of other materials such as metal with sharp edges to facilitate cutting and entry. The devices may further incorporate a rubber dam on the outer catheter to stop blood flow.

The outer diameter of the inner catheter may be substantially the same as the inner diameter of the outer catheter except for a taper at the end of the inner catheter. That is, the inner catheter may have a short taper near the needle. It is preferable that the taper not extend along the length of the exposed portion of the inner catheter. The inner and outer catheters are preferably closely fitted over most of the inner catheter's length. That way, when the outer catheter is advanced into the vein, the vein wall won't catch on the taper of the outer catheter.

There are various common safety devices which may be implemented in the disclosed devices to cover the sharp end of the needle after use. For example, the following U.S. patents, the entirety of which are herein incorporated by reference, disclose various safety devices that may be incorporated: U.S. Pat. Nos. 4,722,344, 4,832,696, 5,000,740, and 5,092,845. The devices may combine the cylinder of the safety device with the hub of the inner catheter.

Other safety devices which may be incorporated include those disclosed in U.S. Pat. No. 6,616,630, the entirety of which is herein incorporated by reference. The clip disclosed therein may be incorporated into the hub of the inner catheter, thus making the catheter and clip both cover the sharp end of the needle. Still other safety devices may be incorporated including one with a spring loaded mechanism to draw the needle into a needle guard.

Various structures may be employed to control movement of the needle and catheters relative to one another. For example, as depicted in FIG. 1, hubs may be used. One or more of the hubs may be replaced with safety devices, tubes, or pegs. Where two hubs are used, both hubs (those of the inner and outer catheters) will fit the tubing which delivers the medication or fluids. Thus, in the event that the catheter is difficult to pass up the vein, the tubing can be connected to the inner catheter and flow started, which will inflate the vein and help “float” the catheters into the vein. After the catheters are fully inserted, the inner catheter can still be withdrawn and the tubing switched to the hub of the outer catheter for normal use.

The following non-limiting example is provided:

STEP 1: A rubber strap or similar device is used to establish a venous tourniquet on the extremity. The patient's arm is positioned to expose a visible or palpable vein. The area of intended insertion is cleaned with betadine or alcohol. As an optional step, an injection of local anesthetic may be made at the site of intended insertion.

STEP 2: The person inserting the catheter uses one hand to stabilize the patient's extremity. The other hand positions the catheter assembly with its tip over the insertion site, angled 30 degrees with the tip toward the skin. The assembly pushed through the skin, with the needle penetrating first and creating a hole for the inner and outer catheters that follow.

STEP 3: When the needle of the assembly penetrates the vein, a visible blood “flash-back” is seen in the flash chamber at the rear end of the needle. After the flash-back, the needle is held still. The inner catheter is advanced into the vein, carrying the outer catheter which remains fixed in position relative to the inner catheter. The needle may be removed now or left in place until the end of the next step.

STEP 4: After the inner catheter has been advanced well into the vein, it is held in a fixed position. The outer catheter is advanced off the inner catheter further into the vein. The outer catheter is advanced into the vein until the hub of the catheter is against the skin.

STEP 5: The needle and inner catheter are removed. The outer catheter is attached to a supply tube for intravenous fluids and medication. The outer catheter is secured by tape or sutures.

In a variation of the procedure above, needle can be removed at the end of step three and the supply tube attached to inner catheter hub. Fluid flow from the supply tube through the inner catheter will inflate the vein and make it easier to pass the inner and outer catheters up the vein. In some versions of the assembly, the inner catheter hub is designed to contain the needle after it is withdrawn. In those versions the supply tube will not connect to the inner catheter, so this variation is not possible.

The technique is the same for arterial insertion, except the tourniquet is not needed.

What is claimed is:

1. A catheter insertion assembly comprising;
a. a needle having a patient insertion end and a second end, a length and a diameter,
b. a first catheter, the first catheter having a patient insertion end and a second end, a length, and an inner diameter sufficient to slidably receive at least a portion of the length of the needle within the first catheter,
c. a second catheter, the second catheter having a patient insertion end and a second end, a length, and an inner diameter sufficient to slidably receive at least a portion of the length of the first catheter within the second catheter, and;
d. wherein the first catheter is circumferentially disposed about at least a portion of the needle, the second catheter is circumferentially disposed about at least a portion of the first catheter, and the first and second catheters are independently slidable from one another and the needle.

2. The catheter assembly of claim 1, wherein the patient insertion end of the needle is distal to the patient insertion end of the first catheter.

3. The catheter assembly of claim 1, wherein the patient insertion end of the first catheter is distal to the patient insertion end of the second catheter.

4. The catheter assembly of claim 1, wherein the patient insertion ends of the needle, first catheter, and second catheter are positioned relative to one another such that when inserting the assembly into a patient, the patient is first contacted by the patient insertion end of the needle, followed by the patient insertion end of the first catheter, followed by the patient insertion end of the second catheter.

5. The catheter assembly of claim 1, wherein the second end of the needle is in fluid communication with a visualization chamber.

6. The catheter assembly of claim 1, further comprising a gripper means attached to the needle for slidable removal of the needle from second catheter after insertion into a patient.

7. The catheter assembly of claim 1, further comprising a detachable hub releasably attached to the second end of the second catheter.

8. The catheter assembly of claim 6, further comprising a safety device attached to the second end of the first catheter capable of receiving the needle upon removal of the needle and the first catheter from the second catheter.

9. The catheter insertion assembly of claim 1, wherein the patient insertion ends of the first and second catheters are tapered.

10. The catheter assembly of claim 1, wherein a separate hub is adjacent to each of the second ends of the needle, first and second catheters.

11. A catheter insertion assembly comprising:
a. a needle having a distal end and a proximal end,
b. a first catheter partially enclosing the needle and having a distal end, a proximal end, and an outer diameter greater than the outer diameter of the needle,
c. a second catheter partially enclosing the needle and the first catheter and having a distal end, a proximal end, and an outer diameter greater than the outer diameter of the first catheter and;
d. wherein the distal end of the needle, and the distal ends of the first and second catheters are configured that upon insertion into a patient, the needle punctures the patient first, followed by the first catheter and then the second catheter, thereby sequentially increasing the diameter of the puncture in the patient.

12. A method for inserting a catheter into a patient comprising:
a. providing a needle having a patient insertion end and a second end, a length and a diameter,
b. providing a first catheter, the first catheter having a patient insertion end and a second end, a length, and an inner diameter sufficient to slidably receive at least a portion of the length of the needle within the first catheter wherein the patient insertion of the needle is distal to the patient insertion end of the first catheter,
c. providing a second catheter, the second catheter having a patient insertion end and a second end, a length, and an inner diameter sufficient to slidably receive at least a portion of the first catheter wherein the patient insertion ends of the needle and the first catheter are distal to the patient insertion end of the second catheter and the first and second catheters are independently slidable from one another and the needle,
d. inserting the patient insertion ends of the needle, first catheter and second catheter into a patient, and;
e. sliding the needle out of the second end of the first catheter.

13. The method of claim 12, further comprising sliding the first catheter out of the second end of the second catheter.

14. The method of claim 13, further comprising leaving the patient insertion end of the second catheter in the patient and attaching a supply catheter to the second catheter.

15. A method for inserting a catheter into a patient comprising
a. providing a needle having a distal end and a proximal end,
b. providing a first catheter partially enclosing the needle and having a distal end, a proximal end, and an outer diameter greater than the outer diameter of the needle,
c. providing a second catheter partially enclosing the needle and the first catheter and having a distal end, a proximal end, and an outer diameter greater than the outer diameter of the first catheter, wherein the distal end of the needle, and the distal ends of the first and second catheters are configured that upon insertion into a patient, the needle punctures the patient first, followed by the first catheter and then the second catheter, thereby sequentially increasing the diameter of the puncture in the patient
d. inserting the distal ends of the needle, first and second catheters into a patient,
e. removing the needle from the first catheter, and;
f. removing the first catheter from the second catheter.

16. The method of claim 15, further comprising leaving the second catheter in the patient and attaching a supply catheter to the second catheter.
